(19) United States
(12) Patent Application Publication
(10) Pub. No.: US 2022/0190038 A1
(43) Pub. Date: Jun. 16, 2022

(54) DETECTION DEVICE

(71) Applicant: Japan Display Inc., Tokyo (JP)

(72) Inventors: Masahiro TADA, Tokyo (JP); Takashi NAKAMURA, Tokyo (JP); Akio TAKIMOTO, Tokyo (JP)

(73) Assignee: Japan Display Inc., Tokyo (JP)

(21) Appl. No.: 17/687,689

(22) Filed: Mar. 7, 2022

Related U.S. Application Data
(63) Continuation of application No. PCT/JP2020/031109, filed on Aug. 18, 2020.

(30) Foreign Application Priority Data
Sep. 12, 2019 (JP) 2019-166577

(51) Int. Cl. H01L 27/28; H01L 31/0475; G01J 1/16; H01L 27/30; H01L 51/42; G06V 40/13

(52) U.S. Cl. CPC H01L 27/286; H01L 31/0475; G01J 1/1626; H01L 31/028; H01L 51/42; G06V 40/1318; H01L 27/301

(57) ABSTRACT

According to an aspect of the present disclosure, a detection device includes: a substrate; a plurality of first optical sensors provided in a detection area of the substrate and comprising an organic material layer having a photovoltaic effect; and at least one or more second optical sensors provided on the substrate and comprising an inorganic material layer having a photovoltaic effect.

### CROSS-REFERENCE TO RELATED APPLICATIONS

This application is a Continuation of International Patent Application No. PCT/JP2020/031109 filed on Aug. 18, 2020, which claims the benefit of priority from Japanese Patent Application No. 2019-166577 filed on Sep. 12, 2019, the entire contents of which are incorporated herein by reference.

### BACKGROUND

### 1. Technical Field

What is disclosed herein relates to a detection device.

### 2. Description of the Related Art

In these years, optical biosensors are known as biosensors used, for example, for personal authentication. Fingerprint sensors (refer to United States Patent Application Publication No. 2018/0012069, for example) and vein sensors are known as such biosensors. Optical sensors using an organic material and optical sensors using an inorganic material are known as optical sensors used as the biosensors.

The optical sensors using an organic material can detect light in a wider wavelength range than the sensors using an inorganic material such as amorphous silicon can. However, the optical sensors using an organic material may change in sensor output due to, for example, aged deterioration.

### SUMMARY

According to an aspect of the present disclosure, a detection device includes: a substrate; a plurality of first optical sensors provided in a detection area of the substrate and comprising an organic material layer having a photovoltaic effect; and at least one or more second optical sensors provided on the substrate and comprising an inorganic material layer having a photovoltaic effect.

### BRIEF DESCRIPTION OF THE DRAWINGS

FIG. 1 is a sectional view illustrating a schematic sectional configuration of a detection apparatus with an illumination device, the detection apparatus including a detection device according to a first embodiment;

FIG. 2 is a plan view illustrating the detection device according to the first embodiment;

FIG. 3 is a block diagram illustrating a configuration example of the detection device according to the first embodiment;

FIG. 4 is a circuit diagram illustrating the detection device;

FIG. 5 is a circuit diagram illustrating a plurality of partial detection areas;

FIG. 6 is a plan view illustrating a first optical sensor;

FIG. 7 is a Q-Q sectional view of FIG. 6;

FIG. 8 is a graph schematically illustrating a relation between a wavelength and a conversion efficiency of light incident on the first optical sensor;

FIG. 9 is a timing waveform diagram illustrating an operation example of the detection device;

FIG. 10 is a timing waveform diagram illustrating an operation example during a reading period in FIG. 9;

FIG. 11 is an XI-XI′ sectional view of FIG. 2;

FIG. 12 is a circuit diagram illustrating a drive circuit of a second optical sensor;

FIG. 13 is an explanatory diagram for explaining a relation between a first detection signal output from the first optical sensor and a second detection signal output from the second optical sensor;

FIG. 14 is a plan view illustrating a detection device according to a second embodiment;

FIG. 15 is a plan view illustrating a detection device according to a third embodiment;

FIG. 16 is a plan view illustrating a detection device according to a fourth embodiment;

FIG. 17 is a XVII-XVII′ sectional view of FIG. 16; and

FIG. 18 is a plan view illustrating a detection device according to a modification of the fourth embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. Moreover, the components described below can be appropriately combined. The disclosure is merely an example, and the present disclosure naturally encompasses appropriate modifications easily conceivable by those skilled in the art while maintaining the gist of the disclosure. To further clarify the description, the drawings schematically illustrate, for example, widths, thicknesses, and shapes of various parts as compared with actual aspects thereof, in some cases. However, they are merely examples, and interpretation of the present disclosure is not limited thereto. The same element as that illustrated in a drawing that has already been discussed is denoted by the same reference numeral through the description and the drawings, and detailed description thereof will not be repeated in some cases where appropriate.

In this disclosure, when an element is described as being “on” another element, the element can be directly on the other element, or there can be one or more elements between the element and the other element.

### First Embodiment

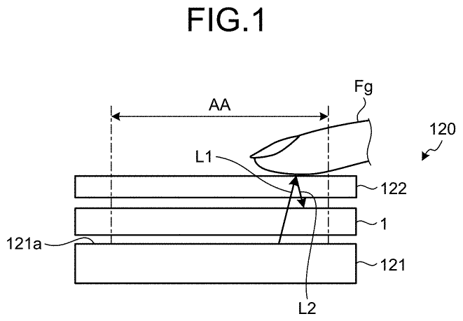

FIG. 1 is a sectional view illustrating a schematic sectional configuration of a detection apparatus with an illumination device, the detection apparatus including a detection device according to a first embodiment. As illustrated in FIG. 1, a detection apparatus 120 with an illumination device includes a detection device 1, an illumination device 121, and a cover glass 122. The illumination device 121, the detection device 1, and the cover glass 122 are stacked in this order in a direction orthogonal to a surface of the detection device 1.

The illumination device 121 has a light-emitting surface 121a and emits light L1 from the light-emitting surface 121a toward the detection device 1. The illumination device 121 is a backlight. The illumination device 121 may be, for example, what is called a side light-type backlight that includes a light guide plate provided at a location corresponding to a detection area AA and a plurality of light sources arranged at one end or both ends of the light guide plate. For example, light-emitting diodes (LEDs) for emitting light in a predetermined color are used as the light sources. The illumination device 121 may be what is called a direct-type backlight that includes light sources (such as LEDs) provided directly below the detection area AA. The illumination device 121 is not limited to the backlight, and may be provided on a lateral side or an upper side of the detection device 1 and emit the light L1 from a lateral side or an upper side of a finger Fg.

The detection device 1 is provided so as to face the light-emitting surface 121a of the illumination device 121. In other words, the detection device 1 is provided between the illumination device 121 and the cover glass 122. The light L1 emitted from the illumination device 121 passes through the detection device 1 and the cover glass 122. The detection device 1 is, for example, an optically reflective biosensor and can detect asperities (such as a fingerprint) on a surface of the finger Fg by detecting light L2 reflected on an interface between the cover glass 122 and air. Alternatively, the detection device 1 may detect biological information, in addition to the fingerprint, by detecting the light L2 reflected in the finger Fg. The biological information is, for example, a blood vessel image of, for example, a vein, pulsation, and/or a pulse wave. The color of the light L1 from the illumination device 121 may be changed depending on a detection target. For example, the illumination device 121 can emit the light L1 in blue or green when detecting the fingerprint, and can emit the infrared light L1 when detecting the vein.

The cover glass 122 is a member for protecting the detection device 1 and the illumination device 121 and covers the detection device 1 and the illumination device 121. The cover glass 122 is, for example, a glass substrate. The cover glass 122 is not limited to a glass substrate and may be, for example, a resin substrate. The cover glass 122 need not be provided. In this case, the surface of the detection device 1 is provided with a protective layer, and the finger Fg contacts the protective layer of the detection device 1.

The detection apparatus 120 with an illumination device may be provided with a display panel instead of the illumination device 121. The display panel may be, for example, an organic electroluminescent (EL) (organic light-emitting diode (OLED)) display panel or an inorganic EL (μ-LED or mini-LED) display panel. Alternatively, the display panel may be a liquid crystal display (LCD) panel using liquid crystal elements as display elements or an electrophoretic display (EPD) panel using electrophoretic elements as the display elements. Also in this case, display light emitted from the display panel passes through the detection device 1, and the fingerprint of the finger Fg and the biological information can be detected based on the light L2 reflected by the finger Fg.

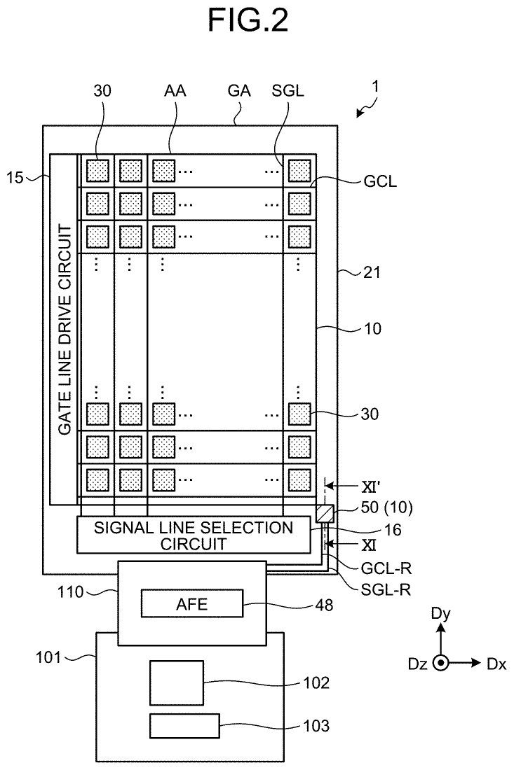

FIG. 2 is a plan view illustrating the detection device according to the first embodiment. As illustrated in FIG. 2, the detection device 1 includes an insulating substrate 21, a sensor 10, a gate line drive circuit 15, a signal line selection circuit 16, a detection circuit 48, a control circuit 102, and a power supply circuit 103.

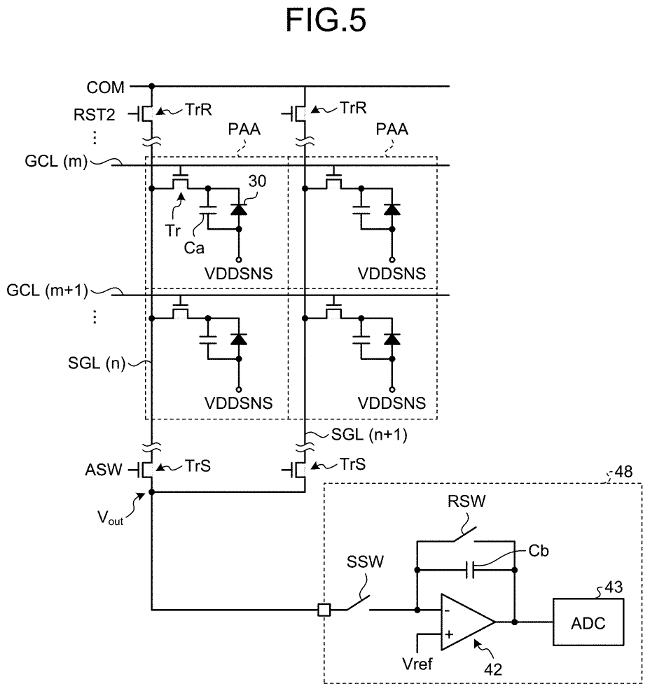

A control board 101 is electrically coupled to the insulating substrate 21 through a flexible printed circuit board 110. The flexible printed circuit board 110 is provided with the detection circuit 48. The control board 101 is provided with the control circuit 102 and the power supply circuit 103. The control circuit 102 is, for example, a field programmable gate array (FPGA). The control circuit 102 supplies control signals to the sensor 10, the gate line drive circuit 15, and the signal line selection circuit 16 to control a detection operation of the sensor 10. The power supply circuit 103 supplies voltage signals including, for example, a sensor power supply signal VDDSNS (refer to FIG. 5) to the sensor 10, the gate line drive circuit 15, and the signal line selection circuit 16.

The insulating substrate 21 has the detection area AA and a peripheral area GA. The detection area AA is an area overlapping a plurality of first optical sensors 30 included in the sensor 10. The peripheral area GA is an area outside the detection area AA and is an area that does not overlap the first optical sensors 30. That is, the peripheral area GA is an area between the outer perimeter of the detection area AA and ends of the insulating substrate 21. The gate line drive circuit 15 and the signal line selection circuit 16 are provided in the peripheral area GA.

The sensor 10 is an optical sensor including the first optical sensors 30 and a second optical sensor 50 that are photoelectric conversion elements. The first optical sensors 30 and the second optical sensor 50 are photodiodes each outputting an electrical signal corresponding to light emitted thereto. The first optical sensors 30 included in the sensor 10 are arranged in a matrix having a row-column configuration in the detection area AA. Each of the first optical sensors 30 outputs an electrical signal corresponding to light emitted thereto as a first detection signal Vdet to the signal line selection circuit 16. The detection device 1 detects the biological information based on the first detection signals Vdet from the first optical sensors 30. In other words, the first optical sensors 30 serve as biosensors. The first optical sensors 30 perform the detection in response to a gate drive signal Vgcl supplied from the gate line drive circuit 15.

The second optical sensor 50 included in the sensor 10 is provided in the peripheral area GA. The second optical sensor 50 is electrically coupled to the detection circuit 48, the control circuit 102, and the power supply circuit 103 through a gate line GCL-R, a signal line SGL-R, and a flexible printed circuit board 110. The second optical sensor 50 outputs an electrical signal corresponding to light emitted thereto as a second detection signal Vdet-R to the detection circuit 48. Based on the second detection signal Vdet-R output from the second optical sensor 50, the control circuit 102 detects changes in the first detection signals Vdet received from the first optical sensors 30 when the same detection target object is detected.

In addition, the control circuit 102 controls the detection in the first optical sensors 30 based on the second detection signal Vdet-R output from the second optical sensor 50 so as to reduce changes in the first detection signals Vdet due to, for example, aged deterioration. In other words, the second optical sensor 50 serves as a reference sensor for the first optical sensors 30. While one second optical sensor 50 is provided in FIG. 2, two or more second optical sensors 50 may be provided.

The gate line drive circuit 15 and the signal line selection circuit 16 are provided in the peripheral area GA. Specifically, the gate line drive circuit 15 is provided in an area of the peripheral area GA extending along a second direction Dy, and the signal line selection circuit 16 is provided in an area of the peripheral area GA extending along a first direction Dx, and is provided between the sensor 10 and the detection circuit 48.

The first direction Dx is a direction in a plane parallel to the insulating substrate 21. The second direction Dy is a direction in a plane parallel to the insulating substrate 21 and is a direction orthogonal to the first direction Dx. The second direction Dy may intersect the first direction Dx without being orthogonal thereto. A third direction Dz is a direction orthogonal to the first direction Dx and the second direction Dy, and is the normal direction of the insulating substrate 21.

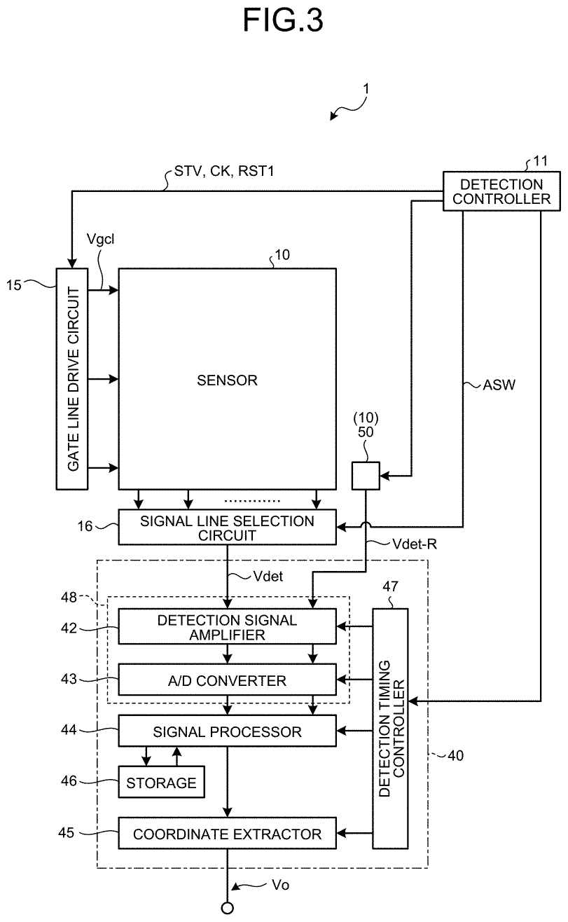

FIG. 3 is a block diagram illustrating a configuration example of the detection device according to the first embodiment. As illustrated in FIG. 3, the detection device 1 further includes a detection controller 11 and a detector 40. The control circuit 102 includes some or all functions of the detection controller 11. The control circuit 102 also includes some or all functions of the detector 40 except those of the detection circuit 48.

The detection controller 11 is a circuit that supplies respective control signals to the gate line drive circuit 15, the signal line selection circuit 16, and the detector 40 to control operations thereof. The detection controller 11 supplies various control signals including, for example, a start signal STV, a clock signal CK, and a reset signal RST1 to the gate line drive circuit 15. The detection controller 11 also supplies various control signals including, for example, a selection signal ASW to the signal line selection circuit 16. The detection controller 11 also supplies control signals to the second optical sensor 50 to control the detection in the second optical sensor 50.

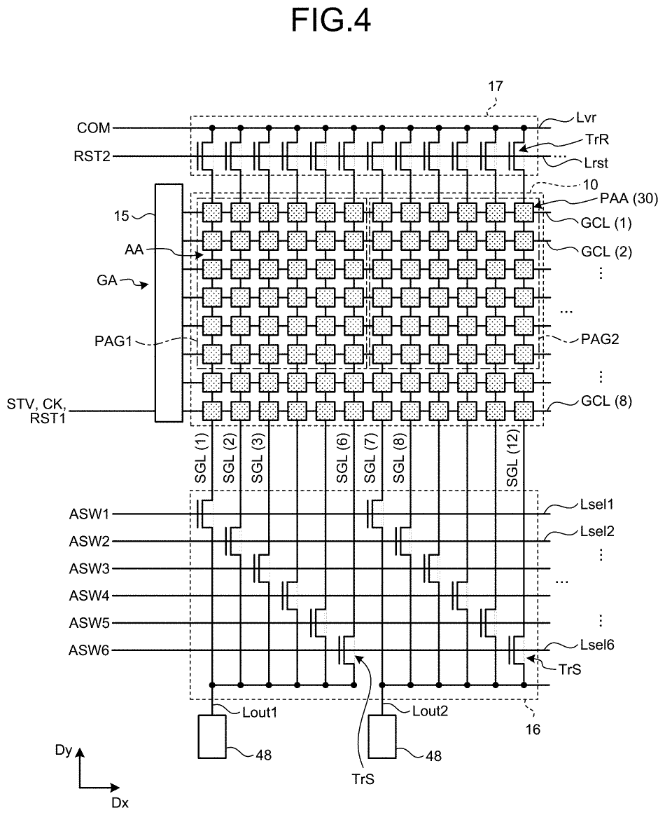

The gate line drive circuit 15 is a circuit that drives a plurality of gate lines GCL (refer to FIG. 4) based on the various control signals. The gate line drive circuit 15 sequentially or simultaneously selects the gate lines GCL and supplies the gate drive signals Vgcl to the selected gate lines GCL. Through this operation, the gate line drive circuit 15 selects the first optical sensors 30 coupled to the gate lines GCL.

The signal line selection circuit 16 is a switch circuit that sequentially or simultaneously selects a plurality of signal lines SGL (refer to FIG. 4). The signal line selection circuit 16 is, for example, a multiplexer. The signal line selection circuit 16 couples the selected signal lines SGL to the detection circuit 48 based on the selection signal ASW supplied from the detection controller 11. Through this operation, the signal line selection circuit 16 outputs the first detection signal Vdet of each of the first optical sensors 30 to the detector 40.

The second optical sensor 50 is driven based on the control signal supplied from the detection controller 11. The second optical sensor 50 outputs the second detection signal Vdet-R to the detector 40 through the signal line SGL-R. The second optical sensor 50 is not coupled to the gate line drive circuit 15 and the signal line selection circuit 16 and is driven independently from the first optical sensors 30. The second optical sensor 50 is not limited to this configuration and may be coupled to the gate line drive circuit 15 and the signal line selection circuit 16. That is, the second optical sensor 50 may be driven based on a drive signal supplied from the gate line drive circuit 15 and may be electrically coupled to the detection circuit 48 through the signal line selection circuit 16.

The detector 40 includes the detection circuit 48, a signal processor 44, a coordinate extractor 45, a storage 46, and a detection timing controller 47. Based on a control signal supplied from the detection controller 11, the detection timing controller 47 controls the detection circuit 48, the signal processor 44, and the coordinate extractor 45 so as to operate in synchronization with one another.

The detection circuit 48 is, for example, an analog front-end (AFE) circuit. The detection circuit 48 is a signal processing circuit having functions of at least a detection signal amplifier 42 and an analog-to-digital (A/D) converter 43. The detection signal amplifier 42 amplifies the first detection signals Vdet and the second detection signal Vdet-R. The A/D converter 43 converts analog signals output from the detection signal amplifier 42 into digital signals.

The signal processor 44 is a logic circuit that detects a predetermined physical quantity received by the sensor 10 based on an output signal of the detection circuit 48. When the finger Fg is in contact with or in proximity to a detection surface, the signal processor 44 can detect the asperities on the surface of the finger Fg or a palm based on the signal from the detection circuit 48. The signal processor 44 can also detect the biological information based on the signal from the detection circuit 48. The biological information is, for example, the blood vessel image, the pulse wave, the pulsation, and/or the blood oxygen saturation level of the finger Fg or the palm. The signal processor 44 calculates a difference signal AV between the first detection signal Vdet and the second detection signal Vdet-R.

The storage 46 temporarily stores therein a signal calculated by the signal processor 44. The storage 46 also stores therein past information on the first detection signals Vdet, the second detection signal Vdet-R, and the difference signals ΔV. The storage 46 may be, for example, a random-access memory (RAM) or a register circuit.

The coordinate extractor 45 is a logic circuit that obtains, when the contact or the proximity of the finger Fg is detected by the signal processor 44, detected coordinates of the asperities on the surface of, for example, a finger Fg. The coordinate extractor 45 is also a logic circuit that obtains detected coordinates of blood vessels of the finger Fg or the palm. The coordinate extractor 45 combines the first detection signals Vdet output from the respective first optical sensors 30 of the sensor 10 to generate two-dimensional information representing the shape of the asperities on the surface of, for example, the finger Fg. The coordinate extractor 45 may output the first detection signals Vdet and the second detection signal Vdet-R as sensor outputs Vo without calculating the detected coordinates.

The following describes a circuit configuration example and an operation example of the detection device 1. FIG. 4 is a circuit diagram illustrating the detection device. FIG. 5 is a circuit diagram illustrating a plurality of partial detection areas. FIG. 5 also illustrates a circuit configuration of the detection circuit 48.

As illustrated in FIG. 4, the sensor 10 has a plurality of partial detection areas PAA arranged in a matrix having a row-column configuration. Each of the partial detection areas PAA is provided with the first optical sensor 30.

The gate lines GCL extend in the first direction Dx and are coupled to the partial detection areas PAA arranged in the first direction Dx. A plurality of gate lines GCL(1), GCL(2), . . . , GCL(8) are arranged in the second direction Dy and are each coupled to the gate line drive circuit 15. In the following description, the gate lines GCL(1), GCL(2), . . . , GCL(8) will each be simply referred to as the gate line GCL when they need not be distinguished from one another. For ease of understanding of the description, FIG. 4 illustrates eight gate lines GCL. However, this is merely an example, and M gate lines GCL (where M is eight or larger, and is, for example, 256) may be arranged.

The signal lines SGL extend in the second direction Dy and are coupled to the first optical sensors 30 of the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when need not be distinguished from one another.

For ease of understanding of the description, 12 signal lines SGL are illustrated. However, this is merely an example, and N signal lines SGL (where N is 12 or larger and is, for example, 252) may be arranged. The resolution of the sensor is, for example, 508 dots per inch (dpi), and the number of cells is 252×256. In FIG. 4, the sensor 10 is provided between the signal line selection circuit 16 and the reset circuit 17. The configuration is not limited thereto. The signal line selection circuit 16 and the reset circuit 17 may be coupled to ends of the signal lines SGL in the same direction.

The gate line drive circuit 15 receives the various control signals such as the start signal STV, the clock signal CK, and the reset signal RST1 from the control circuit 102 (refer to FIG. 2). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and corresponding ones of the partial detection areas PAA arranged in the first direction Dx are selected as detection targets.

The gate line drive circuit 15 may perform different driving for each of detection modes including the detection of a fingerprint and the detection of different items of the biological information (such as the pulse wave, the pulsation, the blood vessel image, and the blood oxygen saturation level). For example, the gate line drive circuit 15 may drive more than one gate line GCL collectively.

Specifically, the gate line drive circuit 15 may simultaneously select a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6), and supplies thereto the gate drive signals Vgcl. The gate line drive circuit 15 supplies the gate drive signals Vgcl through the selected six gate lines GCL to the first switching elements Tr. Through this operation, group areas PAG1 and PAG2 each including more than one partial detection area PAA arranged in the first direction Dx and the second direction Dy are selected as the respective detection targets. The gate line drive circuit 15 drives the predetermined number of the gate lines GCL collectively, and sequentially supplies the gate drive signals Vgcl to the gate lines GCL in units of the predetermined number of the gate lines GCL. Hereinafter, when positions of different group areas such as the group areas PAG1 and PAG2 are not distinguished from each other, each of the group areas will be called “group area PAG”.

The signal line selection circuit 16 includes a plurality of selection signal lines Lsel, a plurality of output signal lines Lout, and third switching elements TrS. The third switching elements TrS are provided so as to correspond to the signal lines SGL. Six signal lines SGL(1), SGL(2), . . . , SGL(6) are coupled to a common output signal line Lout1. Six of the signal lines SGL(7), SGL(8), . . . , SGL(12) are coupled to a common output signal line Lout2. The output signal lines Lout1 and Lout2 are each coupled to the detection circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the third switching elements TrS included in one of the signal line blocks, respectively. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . . . , SGL(6), respectively. The selection signal line Lsel1 is coupled to the third switching element TrS corresponding to the signal line SGL(1) and the third switching element TrS corresponding to the signal line SGL(7). The selection signal line Lsel2 is coupled to the third switching element TrS corresponding to the signal line SGL(2) and the third switching element TrS corresponding to the signal line SGL(8).

The control circuit 102 sequentially supplies the selection signal ASW to the selection signal lines Lsel. Through the operations of the third switching elements TrS, the signal line selection circuit 16 sequentially selects the signal lines SGL in one of the signal line blocks in a time-division manner. The signal line selection circuit 16 selects one of the signal lines SGL in each of the signal line blocks. With the above-described configuration, the detection device 1 can reduce the number of integrated circuits (ICs) including the detection circuit 48 or the number of terminals of the ICs.

The signal line selection circuit 16 may couple more than one signal line SGL to the detection circuit 48 collectively. Specifically, the control circuit 102 simultaneously supplies the selection signal ASW to the selection signal lines Lsel. With this operation, the signal line selection circuit 16 selects, by the operations of the third switching elements TrS, the signal lines SGL (for example, six of the signal lines SGL) in one of the signal line blocks, and couples the signal lines SGL to the detection circuit 48. As a result, signals detected in each group area PAG are output to the detection circuit 48. In this case, signals from the partial detection areas PAA (first optical sensors 30) in each group area PAG are put together and output to the detection circuit 48.

By the operations of the gate line drive circuit 15 and the signal line selection circuit 16, the detection is performed for each group area PAG. As a result, the intensity of the first detection signal Vdet obtained by one time of detection increases, so that the sensor sensitivity can be improved. In addition, time required for the detection can be reduced. Consequently, the detection device 1 can repeatedly perform the detection in a short time, and thus, can improve a signal-to-noise (S/N) ratio, and can accurately detect a change in the biological information with time, such as the pulse wave.

The reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 102 supplies a reset signal RST2 to the reset signal line Lrst. This operation turns on the fourth switching elements TrR to electrically couple the signal lines SGL to the reference signal line Lvr. The power supply circuit 103 supplies a reference signal COM to the reference signal line Lvr. This operation supplies the reference signal COM to a capacitive element Ca (refer to FIG. 5) included in each of the partial detection areas

PAA.

As illustrated in FIG. 5, each of the partial detection areas PAA includes the first optical sensor 30, the capacitive element Ca, and the first switching element Tr. FIG. 5 illustrates two gate lines GCL(m) and GCL(m+1) arranged in the second direction Dy among the gate lines GCL, and illustrates two signal lines SGL(n) and SGL(n+1) arranged in the first direction Dx among the signal lines SGL. The partial detection area PAA is an area surrounded by the gate lines GCL and the signal lines SGL. Each of the first switching elements Tr is provided correspondingly to each of the first optical sensors 30. The first switching element Tr includes a thin-film transistor, and in this example, includes an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the first optical sensor 30 and the capacitive element Ca.

The anode of the first optical sensor 30 is supplied with the sensor power supply signal VDDSNS from the power supply circuit 103. The signal line SGL and the capacitive element Ca are supplied with the reference signal COM that serves as an initial potential of the signal line SGL and the capacitive element Ca from the power supply circuit 103.

When the partial detection area PAA is irradiated with light, a current corresponding to an amount of light flows through the first optical sensor 30. As a result, an electrical charge is stored in the capacitive element Ca. After the first switching element Tr is turned on, a current corresponding to the electrical charge stored in the capacitive element Ca flows through the signal line SGL. The signal line SGL is coupled to the detection circuit 48 through a corresponding one of the third switching elements TrS of the signal line selection circuit 16. Thus, the detection device 1 can detect a signal corresponding to the amount of the light irradiating the first optical sensor 30 in each of the partial detection areas PAA or signals corresponding to the amounts of the light irradiating the first optical sensors 30 in each group area PAG.

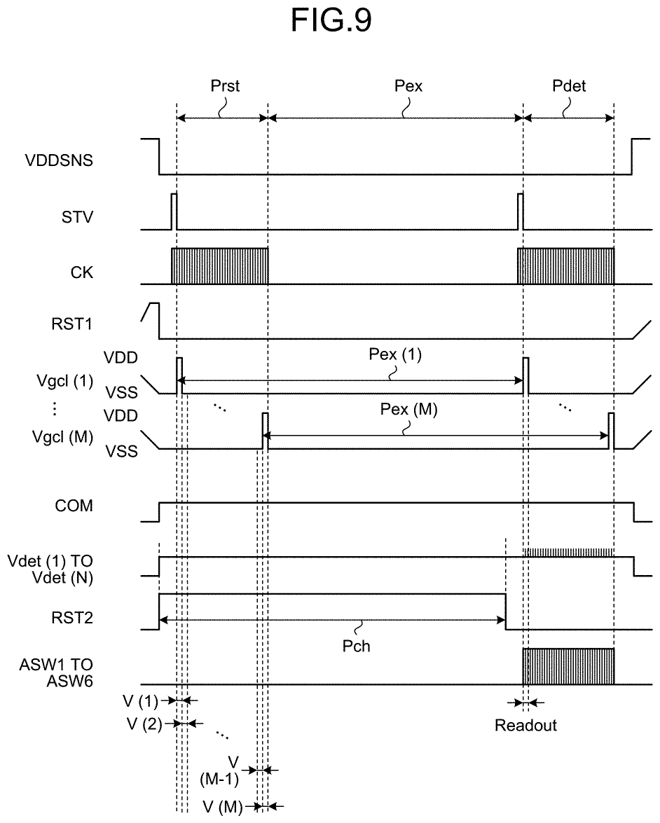

During a reading period Pdet (refer to FIG. 9), a switch SSW of the detection circuit 48 is turned on, and the detection circuit 48 is coupled to the signal lines SGL. The detection signal amplifier 42 of the detection circuit 48 converts a variation of a current supplied from the signal lines SGL into a variation of a voltage, and amplifies the result. A reference potential (Vref) having a fixed potential is supplied to a non-inverting input portion (+) of the detection signal amplifier 42, and the signal lines SGL are coupled to an inverting input portion (−) of the detection signal amplifier 42. The same signal as the reference signal COM is supplied as a reference potential (Vref). The detection signal amplifier 42 includes a capacitive element Cb and a reset switch RSW. During a reset period Prst (refer to FIG. 9), the reset switch RSW is turned on, and an electrical charge of the capacitive element Cb is reset.

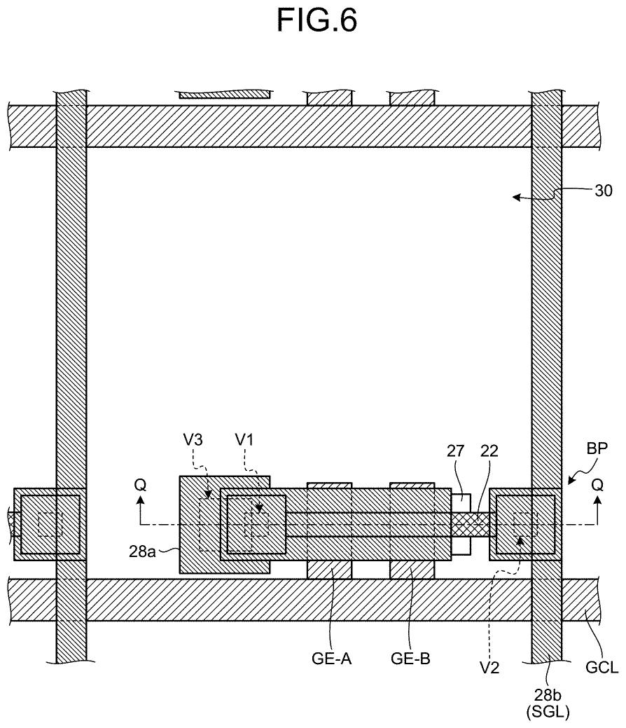

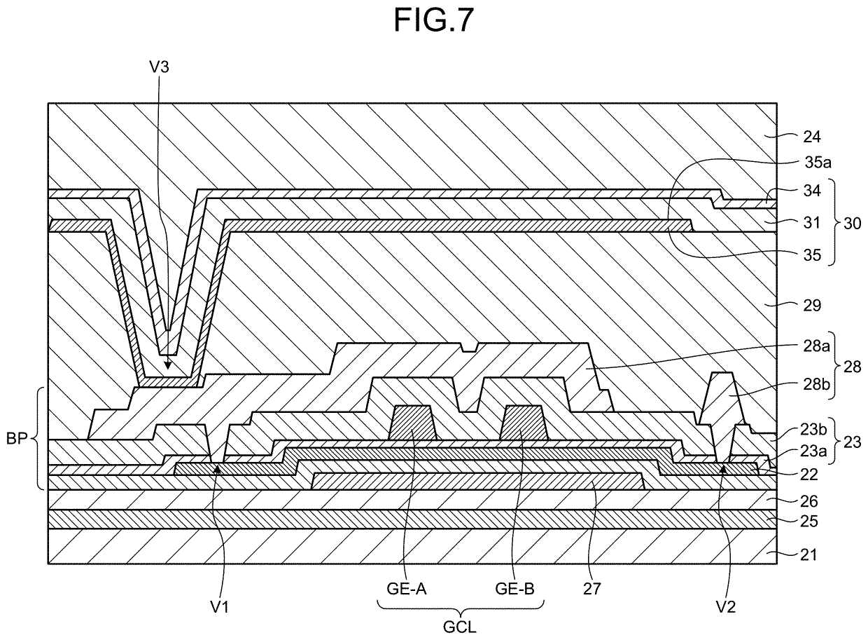

The following describes an outline of a manufacturing method of each of the first optical sensors 30 included in the sensor 10 and a process for forming the first optical sensor 30 (organic photodiode (OPD) forming process). FIG. 6 is a plan view illustrating the first optical sensor. FIG. 7 is a Q-Q sectional view of FIG. 6.

### (Outline of Manufacturing Method)

The outline of the manufacturing method of the first optical sensor 30 included in the sensor 10 will be described. A backplane BP including low-temperature polysilicon (LTPS) 22 is formed on an undercoat 26, a light-blocking layer 27, and an insulator that are stacked on polyimide 25 formed as a film on the insulating substrate 21. The thickness of the polyimide 25 is, for example, 10 μm. A device for forming the backplane BP is separated from the glass substrate using a laser lift-off (LLO) technique after all processes for forming the backplane BP are completed. The backplane BP serves as the first switching elements Tr. While in the present embodiment, the LTPS 22 is employed as a semiconductor layer, the semiconductor layer is not limited thereto, and may be formed of another semiconductor such as amorphous silicon.

Each of the first switching elements Tr includes a double-gate TFT in which two n-channel MOS (NMOS) transistors are coupled. The NMOS transistors of the first switching element Tr have, for example, a channel length of 4.5 μm, a channel width of 2.5 μm, and a mobility of approximately 40 to 70 cm²/Vs. To form the TFT of the LTPS 22, first, four materials of silicon monoxide (SiO), silicon nitride (SiN), SiO, and amorphous silicon (a-Si) are used to form a film, and then, a-Si is annealed by an excimer laser to be crystallized to form polysilicon. A circuit in a surrounding driver portion is formed of a complementary MOS (CMOS) circuit including a p-channel MOS (PMOS) transistor and an NMOS transistor. The PMOS transistor of the surrounding circuit has, for example, a channel length of 4.5 μm, a channel width of 3.5 μm, and a mobility of approximately 40 to 70 cm²/Vs. The NMOS transistor of the surrounding circuit has, for example, a channel length of 4.5 μm, a channel width of 2.5 μm, and a mobility of approximately 40 to 70 cm²/Vs in the same manner as described above. After the polysilicon is formed, electrodes of the PMOS and the NMOS are formed by being doped with boron (B) and phosphorus (P).

Then, SiO is formed as an insulating film 23a, and molybdenum-tungsten alloy (MoW) is formed into films as two gate electrodes GE-A and GE-B of the double-gate TFT. The thickness of the insulating film 23a is, for example, 70 nm. The thickness of MoW for forming the gate electrodes GE-A and GE-B is, for example, 250 nm.

After the MoW films are formed, an intermediate film 23b is formed, and an electrode layer 28 for forming a source electrode 28a and a drain electrode 28b is formed as a film. The electrode layer 28 is, for example, of an aluminum alloy. A via V1 and a via V2 are formed by dry etching that are for coupling the source electrode 28a and the drain electrode 28b to the electrodes of the PMOS and the NMOS of the LTPS 22 that is formed by the doping. The insulating film 23a and the intermediate film 23b serve as an insulating layer 23 that isolates the gate electrodes GE-A and GE-B serving as the gate line GCL from the LTPS 22 and the electrode layer 28.

The thus formed backplane BP includes the LTPS 22 stacked on the first optical sensor 30 side of the light-blocking layer 27, and the electrode layer 28 that is stacked between the LTPS 22 and the first optical sensor 30 and in which the source electrode 28a and the drain electrode 28b of the first switching element Tr are formed. The source electrode 28a extends to a position facing the light-blocking layer 27 with the LTPS 22 interposed therebetween.

After the backplane BP is manufactured, a smooth layer 29 having a thickness of 2 μm is formed to form a layer of an organic photodetector on top of the backplane BP. Although not illustrated, a sealing film is further formed on the smooth layer 29. A via V3 for coupling the backplane BP to the first optical sensor 30 is formed by etching.

Then, an organic photodiode (OPD) having an air-stable inverted structure is formed as the first optical sensor 30 on top of the backplane BP. A material having sensitivity to near-infrared light (for example, light having a wavelength of 850 nm) is used as an active layer 31 (photoelectric conversion layer) of the first optical sensor 30 serving as an organic sensor. Indium tin oxide (ITO) is used as a cathode electrode 35 serving as a transparent electrode, which is coupled to the backplane BP through the via V3. Furthermore, a zinc oxide (ZnO) layer 35a is formed on a surface of ITO to adjust the work function of the electrode.

For the organic photodiode, two different devices are produced using different types of organic semiconductor materials as active layers. Specifically, as the different types of organic semiconductor materials, two types of materials are used, one being PMDPP3T (poly[[2,5-bis(2-hexyldecyl)-2,3,5,6-tetrahydro-3,6-dioxopyrrolo[3,4-c]pyrrole-1,4-diyl]-alt-[3′,3″-dimethyl-2,2′:5′,2″-terthiophene]-5,5″-diyl]), and the other being STD-001 (Sumitomo Chemical Co., Ltd.). A bulk heterostructure is formed by mixing each of the materials with [6,6]-phenyl-C₆₁-butyric acid methyl ester (PCBM), and forming the mixture into a film. Furthermore, a polythiophene-based conductive polymer (PEDOT:PSS) and silver (Ag) are formed into a film as an anode electrode 34. Although not illustrated, the organic photodiode is sealed with parylene having a thickness of 1 μm, and on top of the organic photodiode, chromium and gold (Cr/Au) are formed into a film as a contact pad for coupling to the flexible printed circuit board 110 on which the analog front end (AFE) is mounted.

Although parylene is used as the sealing film, silicon dioxide (SiO₂) or silicon oxynitride (SiON) may be used instead. Although PEDOT:PSS is stacked to 10 nm and Ag is stacked to 80 nm as the anode electrode 34, the range of the film thickness may be from 10 nm to 30 nm for PEDOT:PSS, and from 10 nm to 100 nm for Ag. For example, a molybdenum oxide (MoOx) can be used as an alternative material for PEDOT:PSS. For example, aluminum (Al) or gold (Au) can be used as an alternative material for Ag. Although ZnO is formed on ITO of the cathode electrode 35, a polymer such as polyethylenimine (PEI) or ethoxylated PEI (PEIE) may be formed on ITO.

### (OPD Forming Process)

A surface of the chip is subjected to an O₂ plasma treatment under the condition of 300 W for 10 seconds. Then, the ZnO layer is formed as a film under the spin-coating condition of 5000 rpm for 30 seconds and is annealed at 180° C. for 30 minutes. A PMDPP3T:PCBM solution or a STD-001:PCBM solution is spin-coated as an organic layer on the surface of ZnO at 250 rpm for 4 minutes. Then, a solution obtained by diluting PEDOT:PSS (for example, Al4083) with isopropyl alcohol (IPA) to (3:17) under a nitrogen atmosphere is filtered through a polyvinylidene fluoride (PVDF) filter of 0.45 μm, and then, is formed into a film using the spin-coating method under the condition of 2000 rpm for 30 seconds (sec). After the film formation, annealing is performed at 80° C. for 5 minutes (min) under the nitrogen atmosphere. Finally, silver is vacuum-deposited to 80 nm as the anode electrode 34. After the device is completed, parylene is formed into a film of 1 μm as the sealing film using a chemical vapor deposition (CVD) method, and Cr/Au is vacuum-deposited as the contact pad.

The first optical sensor 30 formed by such a forming process includes the active layer 31 serving as an organic material layer having a photovoltaic effect, the cathode electrode 35 provided on a side of the active layer 31 closer to the backplane BP, and the anode electrode 34 provided on a side of the active layer 31 opposite to the cathode electrode 35. The layer of the active layer 31 and the layer of the anode electrode 34 are continuous along a detection surface of the sensor 10 provided so as to be capable of detecting the light, over the cathode electrodes 35 of each of the first optical sensors 30 arranged along the detection surface of the sensor 10 (refer to FIG. 7). That is, the cathode electrode 35 is provided independently for each of the first optical sensors 30, and the active layer 31 and the anode electrode 34 are continuous over the entire detection area AA.

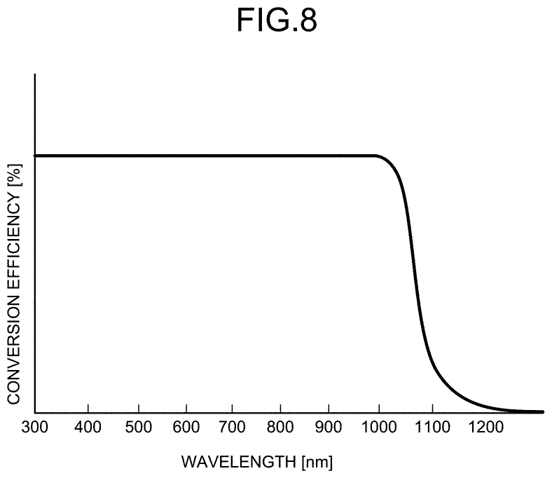

FIG. 8 is a graph schematically illustrating a relation between the wavelength and a conversion efficiency of light incident on the first optical sensor. The horizontal axis of the graph illustrated in FIG. 8 represents the wavelength of the light incident on the first optical sensor 30, and the vertical axis of the graph represents an external quantum efficiency of the first optical sensor 30. The external quantum efficiency is expressed as a ratio between the number of photons of the light incident on the first optical sensor 30 and a current that flows from the first optical sensor 30 to the external detection circuit 48.

As illustrated in FIG. 8, the first optical sensor 30 has a good efficiency in a wavelength band from approximately 300 nm to approximately 1000 nm. That is, the first optical sensor 30 has a sensitivity, for example, from a wavelength range of visible light to a wavelength range of infrared light. Therefore, each of the first optical sensors 30 can detect a plurality of beams of light having different wavelengths even when the illumination device 121 emits the light L1 having wavelength ranges different depending on detection targets.

The following describes an operation example of the detection device 1. FIG. 9 is a timing waveform diagram illustrating the operation example of the detection device. As illustrated in FIG. 9, the detection device 1 has the reset period Prst, an effective exposure period Pex, and the reading period Pdet. The power supply circuit 103 supplies the sensor power supply signal VDDSNS to the anode of the first optical sensor 30 over the reset period Prst, the effective exposure period Pex, and the reading period Pdet. The sensor power supply signal VDDSNS is a signal for applying a reverse bias between the anode and the cathode of the first optical sensor 30. For example, the reference signal COM of substantially 0.75 V is applied to the cathode of the first optical sensor 30, and the sensor power supply signal VDDSNS of substantially −1.25 V is applied to the anode. As a result, a reverse bias of substantially 2.0 V is applied between the anode and the cathode.

The control circuit 102 sets the reset signal RST2 to “H”, and then, supplies the start signal STV and the clock signal CK to the gate line drive circuit 15 to start the reset period Prst. During the reset period Prst, the control circuit 102 supplies the reference signal COM to the reset circuit 17 and uses the reset signal RST2 to turn on the fourth switching elements TrR for supplying a reset voltage. This operation supplies the reference signal COM as the reset voltage to each of the signal lines SGL. The reference signal COM is set to, for example, 0.75 V.

During the reset period Prst, the gate line drive circuit 15 sequentially selects each of the gate lines GCL based on the start signal STV, the clock signal CK, and the reset signal RST1. The gate line drive circuit 15 sequentially supplies the gate drive signals Vgcl {Vgcl(1), . . . , Vgcl(M)} to the gate lines GCL. The gate drive signal Vgcl has a pulsed waveform having a power supply voltage VDD serving as a high-level voltage and a power supply voltage VSS serving as a low-level voltage. In FIG. 9, M gate lines GCL (where M is, for example, 256) are provided, and the gate drive signals Vgcl(1), . . . , Vgcl(M) are sequentially supplied to the respective gate lines GCL. Thus, the first switching elements Tr are sequentially brought into a conducting state and supplied with the reset voltage on a row-by-row basis. For example, a voltage of 0.75 V of the reference signal COM is supplied as the reset voltage.

Thus, during the reset period Prst, the capacitive elements Ca of all the partial detection areas PAA are sequentially electrically coupled to the signal lines SGL, and are supplied with the reference signal COM. As a result, the capacitance of the capacitive elements Ca is reset. The capacitance of the capacitive elements Ca of some of the partial detection areas PAA can be reset by partially selecting the gate lines GCL and the signal lines SGL.

Examples of a control method of the exposure timing include a gate line scanning time exposure control method and a full-time exposure control method. In the gate line scanning time exposure control method, the gate drive signals Vgcl(1), . . . , Vgcl(M) are sequentially supplied to all the respective gate lines GCL coupled to the first optical sensors 30 of the detection targets, and all the first optical sensors 30 of the detection targets are supplied with the reset voltage. Then, after all the gate lines GCL coupled to the first optical sensors 30 of the detection targets are set to a low voltage (the first switching elements Tr are turned off), the exposure starts, whereby the exposure is performed during the effective exposure period Pex. After the exposure ends, the gate drive signals Vgcl(1), . . . , Vgcl(M) are sequentially supplied to the gate lines GCL coupled to the first optical sensors 30 of the detection targets as described above, and reading is performed during the reading period Pdet.

In the full-time exposure control method, control for performing the exposure can also be performed during the reset period Prst and the reading period Pdet (full-time exposure control). In this case, the effective exposure period Pex(1) starts after the gate drive signal Vgcl(M) is supplied to the gate line GCL. The term “effective exposure periods Pex(1), . . . , Pex(M)” refers to a period during which the capacitive elements Ca are charged from the first optical sensors 30.

The start timing and the end timing of the actual effective exposure periods Pex(1), . . . , Pex(M) are different among the partial detection areas PAA corresponding to the gate lines GCL. Each of the effective exposure periods Pex(1), . . . , Pex(M) starts when the gate drive signal Vgcl changes from the power supply voltage VDD serving as the high-level voltage to the power supply voltage VSS serving as the low-level voltage during the reset period Prst. Each of the effective exposure periods Pex(1), . . . , Pex(M) ends when the gate drive signal Vgcl changes from the power supply voltage VSS to the power supply voltage VDD during the reading period Pdet. The lengths of the exposure time of the effective exposure periods Pex(1), . . . , Pex(M) are equal.

In the gate line scanning time exposure control method, a current flows depending on the light irradiating the first optical sensor 30 in each of the partial detection areas PAA during the effective exposure period Pex. As a result, an electrical charge is stored in each of the capacitive elements Ca.

At a time before the reading period Pdet starts, the control circuit 102 sets the reset signal RST2 to a low-level voltage. This operation stops operation of the reset circuit 17. The reset signal may be set to a high-level voltage only during the reset period Prst. During the reading period Pdet, the gate line drive circuit 15 sequentially supplies the gate drive signals Vgcl(1) . . . , Vgcl(M) to the gate lines GCL in the same manner as during the reset period Prst.

Specifically, the gate line drive circuit 15 supplies the gate drive signal Vgcl(1) at the high-level voltage (power supply voltage VDD) to the gate line GCL(1) during a period V(1). The control circuit 102 sequentially supplies the selection signals ASW1, . . . , ASW6 to the signal line selection circuit 16 during a period in which the gate drive signal Vgcl(1) is at the high-level voltage (power supply voltage VDD). This operation sequentially or simultaneously couples the signal lines SGL of the partial detection areas PAA selected by the gate drive signal Vgcl(1) to the detection circuit 48. As a result, the first detection signal Vdet for each of the partial detection areas PAA is supplied to the detection circuit 48.

In the same manner, the gate line drive circuit 15 supplies the gate drive signals Vgcl(2), . . . , Vgcl(M−1), Vgcl(M) at the high-level voltage to gate lines GCL(2), . . . , GCL(M−1), GCL(M) during periods V(2), . . . , V(M−1), V(M), respectively. That is, the gate line drive circuit 15 supplies the gate drive signal Vgcl to the gate line GCL during each of the periods V(1), V(2), . . . , V(M−1), V(M). The signal line selection circuit 16 sequentially selects each of the signal lines SGL based on the selection signal ASW in each period in which the gate drive signal Vgcl is set to the high-level voltage. The signal line selection circuit 16 sequentially couples each of the signal lines SGL to one detection circuit 48. Thus, the detection device 1 can output the first detection signals Vdet of all the partial detection areas PAA to the detection circuit 48 during the reading period Pdet.

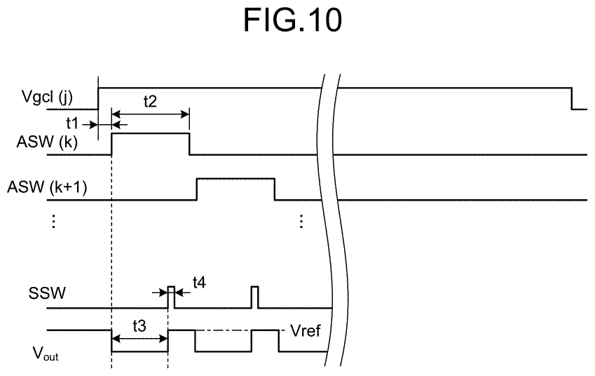

FIG. 10 is a timing waveform diagram illustrating an operation example during a reading period in FIG. 9. With reference to FIG. 10, the following describes the operation example during a supply period Readout of one of the gate drive signals Vgcl(j) in FIG. 9. In FIG. 9, the reference sign of the supply period “Readout” is assigned to the first gate drive signal Vgcl(1), and the same applies to the other gate drive signals Vgcl(2) . . . , Vgcl(M). The index j is any one of the natural numbers 1 to M.

As illustrated in FIGS. 10 and 5, an output (Vₒᵤₜ) of each of the third switching elements TrS has been reset to the reference potential (Vref) in advance. The reference potential (Vref) serves as a reset voltage, and is set to, for example, 0.75 V. Then, the gate drive signal Vgcl(j) is set to a high level, and the first switching elements Tr of a corresponding row are turned on. Thus, each of the signal lines SGL of each row is set to a voltage corresponding to the electrical charge stored in the capacitive element Ca of the partial detection area PAA.

After a period t1 elapses from a rise of the gate drive signal Vgcl(j), a period t2 starts in which the selection signal ASW(k) is set to a high level. After the selection signal ASW(k) is set to the high level and the third switching element TrS is turned on, the output (Vₒᵤₜ) of the third switching element TrS (refer to FIG. 5) is changed to a voltage corresponding to the electrical charge stored in the capacitive element Ca of the partial detection area PAA coupled to the detection circuit 48 through the third switching element TrS, by the electrical charge stored in the capacitive element Ca (period t3).

In the example of FIG. 10, this voltage is reduced from the reset voltage as illustrated in the period t3. Then, after the switch SSW is turned on (period t4 during which an SSW signal is set to a high-level), the electrical charge stored in the capacitive element Ca moves to a capacitive element Cb of the detection signal amplifier 42 of the detection circuit 48, and the output voltage of the detection signal amplifier 42 is set to a voltage corresponding to the electrical charge stored in the capacitive element Cb. At this time, the potential of an inverting input portion of the detection signal amplifier 42 is set to an imaginary short-circuit potential of the operational amplifier, and therefore, returns to the reference potential (Vref).

The A/D converter 43 reads the output voltage of the detection signal amplifier 42. In the example of FIG. 10, waveforms of the selection signals ASW(k), ASW(k+1), . . . corresponding to the signal lines SGL of the respective columns are set to a high level to sequentially turn on the third switching elements TrS. The same operation is sequentially performed, thereby sequentially reading the electrical charges stored in the capacitive elements Ca of the partial detection areas PAA coupled to the gate line GCL. ASW(k), ASW(k+1), . . . in FIG. 10 are, for example, any of ASW 1 to ASW 6 in FIG. 9.

Specifically, after the period t4 starts in which the switch SSW is on, the electrical charge moves from the capacitive element Ca of the partial detection area PAA to the capacitive element Cb of the detection signal amplifier 42 of the detection circuit 48. At this time, the non-inverting input (+) of the detection signal amplifier 42 is biased to the reference potential (Vref) (for example, 0.75 V). As a result, the output (Vₒᵤₜ) of the third switching element TrS is also set to the reference potential (Vref) due to the imaginary short-circuit between input ends of the detection signal amplifier 42.

The voltage of the capacitive element Cb is set to a voltage corresponding to the electrical charge stored in the capacitive element Ca of the partial detection area PAA at a location where the third switching element TrS is turned on in response to the selection signal ASW(k). After the output (Vₒᵤₜ) of the third switching element TrS is set to the reference potential (Vref) due to the imaginary short-circuit, the output of the detection signal amplifier 42 reaches a capacitance corresponding to the voltage of the capacitive element Cb, and this output voltage is read by the A/D converter 43. The voltage of the capacitive element Cb is, for example, a voltage between two electrodes in a capacitor constituting the capacitive element Cb.

The period t1 is, for example, 20 μs. The period t2 is, for example, 60 μs. The period t3 is, for example, 44.7 μs. The period t4 is, for example, 0.98 μs.

Although FIGS. 9 and 10 illustrate the example in which the gate line drive circuit 15 selects the gate line GCL individually, the number of the gate lines GCL to be selected is not limited to this example. The gate line drive circuit 15 may simultaneously select a predetermined number (two or more) of the gate lines GCL and sequentially supply the gate drive signals Vgcl to the gate lines GCL in units of the predetermined number of the gate lines GCL. The signal line selection circuit 16 may also simultaneously couple a predetermined number (two or more) of the signal lines SGL to one detection circuit 48. Moreover, the gate line drive circuit 15 may skip some of the gate lines GCL and scan the remaining ones.

The detection device 1 can detect a fingerprint based on capacitance. Specifically, the capacitive element Ca is used. First, all the capacitive elements Ca are each charged with a predetermined electrical charge. Then, a finger Fg touches the detection area AA, and thereby, capacitance corresponding to the asperities of the fingerprint is added to the capacitive element Ca of each of the cells. Thus, a fingerprint pattern can be generated by allowing the detection signal amplifier 42 and the A/D converter 43 to read the capacitance indicated by the output from the capacitive element Ca of each of the cells in the state where the finger Fg is in contact with the detection area AA, in the same manner as the acquisition of the output from each of the partial detection areas PAA described with reference to FIGS. 9 and 10. This method allows a fingerprint to be detected using a capacitance method. A structure is preferably employed in which the distance between the capacitor of the partial detection area PAA and a n object to be detected such as a fingerprint is set in a range from 100 μm to 300 μm.

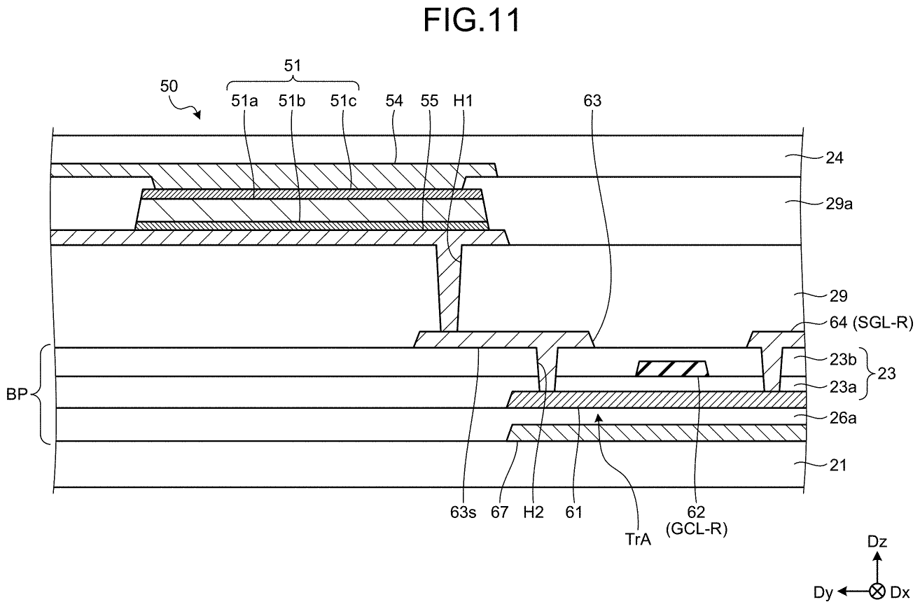

The following describes a configuration of the second optical sensor 50. FIG. 11 is an XI-XI403 sectional view of FIG. 2. As illustrated in FIG. 11, the second optical sensor 50 is provided above the same insulating substrate 21 as in the case of the first optical sensor 30. More specifically, the second optical sensor 50 is provided on the smooth layer 29.

The second optical sensor 50 includes an inorganic material layer (semiconductor layer 51) having a photovoltaic effect. Specifically, the second optical sensor 50 includes the semiconductor layer 51, an anode electrode 54, and a cathode electrode 55. The cathode electrode 55, the semiconductor layer 51, and the anode electrode 54 are stacked in this order on the smooth layer 29. The semiconductor layer 51 is an inorganic semiconductor layer formed of, for example, amorphous silicon (a-Si). The semiconductor layer 51 is not limited to being formed of amorphous silicon, and may be formed of, for example, polysilicon, or more preferably, LTPS.

The second optical sensor 50 is, for example, a positive-intrinsic-negative (PIN) photodiode. Specifically, the semiconductor layer 51 includes an i-type semiconductor layer 51a, an n-type semiconductor layer 51b, and a p-type semiconductor layer 51c. The i-type semiconductor layer 51a, the n-type semiconductor layer 51b, and the p-type semiconductor layer 51c constitute a specific example of the photoelectric conversion element. In FIG. 11, the i-type semiconductor layer 51a is provided between the n-type semiconductor layer 51b and the p-type semiconductor layer 51c in a direction (third direction Dz) orthogonal to a surface of the insulating substrate 21. In the present embodiment, the n-type semiconductor layer 51b, the i-type semiconductor layer 51a, and the p-type semiconductor layer 51c are stacked in this order above the cathode electrode 55.

The a-Si of the p-type semiconductor layer 51c is doped with impurities to form an n+ region. The a-Si of the n-type semiconductor layer 51b is doped with impurities to form a p+ region. The i-type semiconductor layer 51a is, for example, a non-doped intrinsic semiconductor, and has lower conductivity than those of the p-type semiconductor layer 51c and the n-type semiconductor layer 51b.

The anode electrode 54 and the cathode electrode 55 are formed of a light-transmitting conductive material such as indium tin oxide (ITO). The anode electrode 54 is an electrode for supplying the sensor power supply signal to the photoelectric conversion layer. The cathode electrode 55 is an electrode for reading the second detection signal Vdet-R.

The anode electrode 54 is provided on a smooth layer 29a. The smooth layer 29a is provided with an opening in an area overlapping the semiconductor layer 51. The anode electrode 54 is coupled to the semiconductor layer 51 through the opening of the smooth layer 29a. The cathode electrode 55 is provided on the smooth layer 29. The cathode electrode 55 is coupled to the backplane BP through a contact hole H1 passing through the smooth layer 29.

A fifth switching element TrA coupled to the second optical sensor 50 includes a semiconductor layer 61, a gate electrode 62, a source electrode 63, and a drain electrode 64. A light-blocking film 67 is provided between the semiconductor layer 61 and the insulating substrate 21. The cathode electrode 55 of the second optical sensor 50 is coupled to the source electrode 63 through coupling wiring 63s. The sectional structure of the fifth switching element TrA is the same as that of the first switching element Tr described above with reference to FIG. 7, and therefore, will not be described in detail. The fifth switching element TrA is not limited to the case of being provided in the same layer as that of the first switching element Tr, and may be formed in a layer different from that of the first switching element Tr.

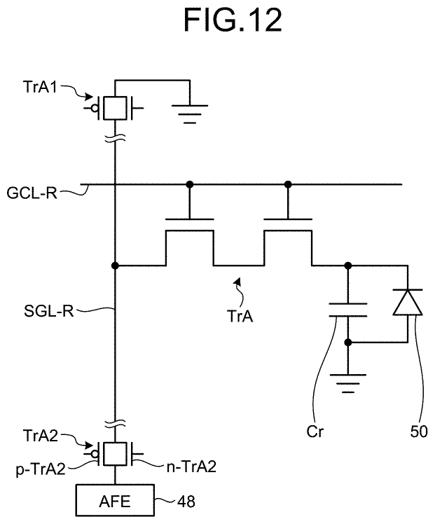

FIG. 12 is a circuit diagram illustrating a drive circuit of the second optical sensor. As illustrated in FIG. 12, the gates of the fifth switching element TrA are coupled to the gate line GCL-R. The source of the fifth switching element TrA is coupled to the signal line SGL-R. The drain of the fifth switching element TrA is coupled to the cathode electrode 55 of the second optical sensor 50 and one end of a capacitive element Cr. The anode electrode 54 of the second optical sensor 50 and the other end of the capacitive element Cr are coupled to the reference potential such as a ground potential.

A sixth switching element TrA1 and a seventh switching element TrA2 are coupled to the signal line SGL-R. The sixth switching element TrA1 and the seventh switching element TrA2 are elements included in a drive circuit for driving the fifth switching element TrA. Each of the sixth switching element TrA1 and the seventh switching element TrA2 is fabricated from, for example, a complementary MOS (CMOS) transistor obtained by combining a p-channel transistor p-TrA2 with an n-channel transistor n-TrA2.

In the present embodiment, the drive circuit of the second optical sensor 50 is provided in the peripheral area GA. The drive circuit of the second optical sensor 50 is provided separately from the gate line drive circuit 15 and the signal line selection circuit 16, and the control circuit 102 can drive the second optical sensor 50 independently from the first optical sensors 30. However, the gate line drive circuit 15 and the signal line selection circuit 16 may be commonly used also as the drive circuit of the second optical sensor 50. The control circuit 102 may drive the second optical sensor 50 in synchronization with the first optical sensors 30.

When the second optical sensor 50 is irradiated with light, a current corresponding to the amount of the light flows through the second optical sensor 50, whereby an electrical charge is stored in the capacitive element Cr. After the fifth switching element TrA is turned on, a current corresponding to the electrical charge stored in the capacitive element Cr flows through the signal line SGL-R. The signal line SGL-R is coupled to the detection circuit 48 through the seventh switching element TrA2. Thus, the detection device 1 can detect a signal corresponding to the amount of the light irradiating the second optical sensor 50 as the second detection signal Vdet-R. The driving method (the reset period Prst, the effective exposure period Pex, and the reading period Pdet) of the second optical sensor 50 is the same as that of the partial detection area PAA of the first optical sensor 30 described above, and thus, will not be described in detail.

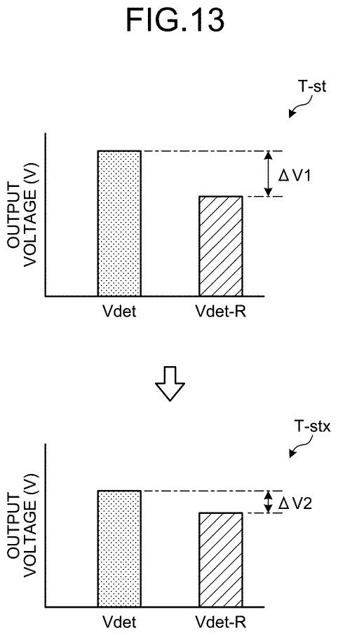

FIG. 13 is an explanatory diagram for explaining a relation between the first detection signal output from the first optical sensor and the second detection signal output from the second optical sensor. As illustrated in FIG. 13, the detection device 1 simultaneously drives the first optical sensors 30 and the second optical sensor 50 at a first time point T-st. The first detection signal Vdet and the second detection signal Vdet-R at the first time point T-st are detection signals of the same detection target object (such as a finger Fg) obtained when the detection target object is detected by the first optical sensors 30 and the second optical sensor 50, respectively. The first detection signal Vdet may be the individual first detection signal Vdet output from each of the first optical sensors 30, or may be the average value of the first detection signals Vdet.

The signal processor 44 calculates a difference signal ΔV1 between the first detection signal Vdet and the second detection signal Vdet-R at the first time point T-st. The difference signal ΔV1 is stored in the storage 46. The first time point T-st is, for example, the time of the start-up of the detection device 1, and the examples thereof include the time when the power supply is turned from off to on and the time when the detection device 1 is returned from a sleep mode.

At a second time point T-stx after a predetermined period has elapsed from the first time point T-st, the detection device 1 simultaneously drives the first optical sensors 30 and the second optical sensor 50. The signal processor 44 calculates a difference signal ΔV2 between the first detection signal Vdet and the second detection signal Vdet-R at the second time point T-stx.

The control circuit 102 compares the difference signal ΔV2 with the difference signal ΔV1 to calculate a difference ΔV3 (=|ΔV2−ΔV1|) between the difference signal ΔV2 and the difference signal ΔV1. If the difference ΔV3 is equal to or larger than a predetermined value, the control circuit 102 determines that the first detection signal Vdet has changed due to, for example, change in the first optical sensors 30 with time, even when the same detection target object is detected under the same condition.

When the first detection signal Vdet has changed, the control circuit 102 changes the drive condition of the first optical sensors 30 so as to reduce the difference ΔV3 to be smaller than the predetermined value, that is, so as to make the difference signal ΔV2 closer to the difference signal ΔV1. For example, the control circuit 102 can adjust the first detection signals Vdet by changing the sensor power supply signal VDDSNS of the first optical sensors 30, or by changing the length of the effective exposure period Pex. Alternatively, the control circuit 102 may cause the signal processor 44 to adjust the digital data supplied from the A/D converter 43.

For ease of understanding of the description, FIG. 13 illustrates the detection signals at the first time point T-st and the second time point T-stx. However, the detection device 1 may drive the second optical sensor 50 in any manner. For example, the detection device 1 may drive the second optical sensor 50 such that the second optical sensor 50 operates in full-time synchronization with the first optical sensors 30. Alternatively, the detection device 1 may drive the second optical sensor 50 each time of the start-up of the detection device 1, or may drive the second optical sensor 50 once in one frame period or once in a plurality of frame periods, where the one frame period is defined as a period in which the first optical sensors 30 perform the detection in the entire detection area AA.

As described above, the detection device 1 of the present embodiment includes the substrate (insulating substrate 21), the first optical sensors 30, and at least one or more of the second optical sensors 50. The first optical sensors 30 are provided in the detection area AA of the substrate and each include the organic material layer (active layer 31) having a photovoltaic effect. The second optical sensor 50 is provided on the substrate and includes the inorganic material layer (semiconductor layer 51) having a photovoltaic effect.

Even when the first detection signals Vdet change due to, for example, aged deterioration of the first optical sensors 30 using the organic material, the second optical sensor 50 using the inorganic material is subjected to a smaller change with time than the first optical sensors 30 are. That is, the change in the second detection signal Vdet-R with time is much smaller than the changes in the first detection signals Vdet with time. As a result, the detection device 1 can detect the changes in the first detection signals Vdet using, as a reference, the second detection signal Vdet-R from the second optical sensor 50 using the inorganic material. The detection device 1 can reduce the changes in the first detection signals Vdet by adjusting the drive of the first optical sensors 30 and by adjusting the signal processing by the detector 40. As a result, the detection device 1 can reduce the degradation in the detection performance.

In the detection device 1, the first optical sensors 30 are arranged in a matrix having a row-column configuration in the detection area AA, and one second optical sensor 50 is disposed in the peripheral area GA of the substrate. This configuration can make the resolution of the detection higher than in a case where the second optical sensor 50 is provided in the detection area AA. Since the one second optical sensor 50 is disposed, the circuit scale of the surrounding circuit provided in the peripheral area GA can be reduced.

In, for example, FIG. 2, the first optical sensors 30 and the second optical sensor 50 have substantially a quadrilateral shape in a plan view. However, the first optical sensors 30 and the second optical sensor 50 are not limited to this shape and may have another shape such as a polygonal shape or a circular shape. The circuits for driving the first optical sensors 30 illustrated in FIGS. 4 and 5 and the second optical sensor 50 illustrated in FIG. 12 are merely examples and can be changed as appropriate.

### Second Embodiment

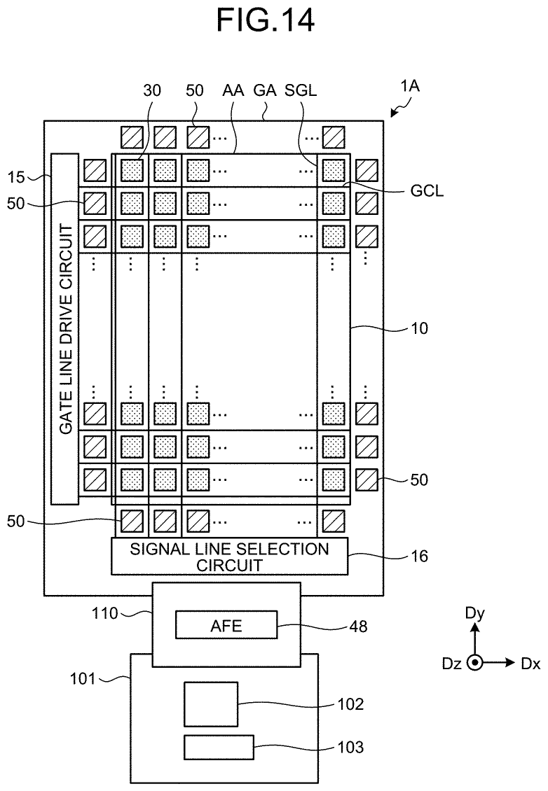

FIG. 14 is a plan view illustrating a detection device according to a second embodiment. The same components as those described in the above-described first embodiment are denoted by the same reference numerals, and the description thereof will not be repeated. As illustrated in FIG. 14, a detection device 1A of the second embodiment includes a plurality of the second optical sensors 50.

The second optical sensors 50 are provided in the peripheral area GA and are arranged along at least one side of the detection area AA. More specifically, the second optical sensors 50 are arranged in a frame shape so as to surround the detection area AA. The second optical sensors 50 are provided between the gate line drive circuit 15 and the detection area AA. The second optical sensors 50 are also provided between the signal line selection circuit 16 and the detection area AA.

The gate line GCL-R coupled to the second optical sensor 50 (refer to FIG. 12) may be coupled to the gate line drive circuit 15. The signal line SGL-R coupled to the second optical sensor 50 (refer to FIG. 12) may be coupled to the signal line selection circuit 16.

In the present embodiment, the control circuit 102 can compare the first detection signal Vdet and the second detection signal Vdet-R output from the first optical sensor 30 and the second optical sensor 50 arranged close to each other. For example, the control circuit 102 can divide the detection area AA and the peripheral area GA into a plurality of areas, and compare the first detection signal Vdet with the second detection signal Vdet-R on an area-by-area basis.

The detection device 1A can compare the first optical sensor 30 and the second optical sensor 50 arranged close to each other, and therefore, can accurately detect the change in the first detection signal Vdet due to, for example, the change in the first optical sensor 30 with time. The control circuit 102 may calculate the average value of the second detection signals Vdet-R output from the second optical sensors 50 and may use the average value of the second detection signals Vdet-R as a reference for the first detection signal Vdet.

The arrangement of the second optical sensors 50 is not limited to the example illustrated in FIG. 14. For example, the second optical sensors 50 are not limited to having the configuration of surrounding the four sides of the detection area AA and need not be provided along one side of the detection area AA. The arrangement pitch of the second optical sensors 50 and the arrangement pitch of the first optical sensors 30 are the same as each other, but may differ from each other. That is, the number of the second optical sensors 50 arranged along the second direction Dy may differ from the number of the first optical sensors 30 arranged along the second direction Dy. Also, the number of the second optical sensors 50 arranged along the first direction Dx may differ from the number of the first optical sensors 30 arranged along the first direction Dx.

### Third Embodiment

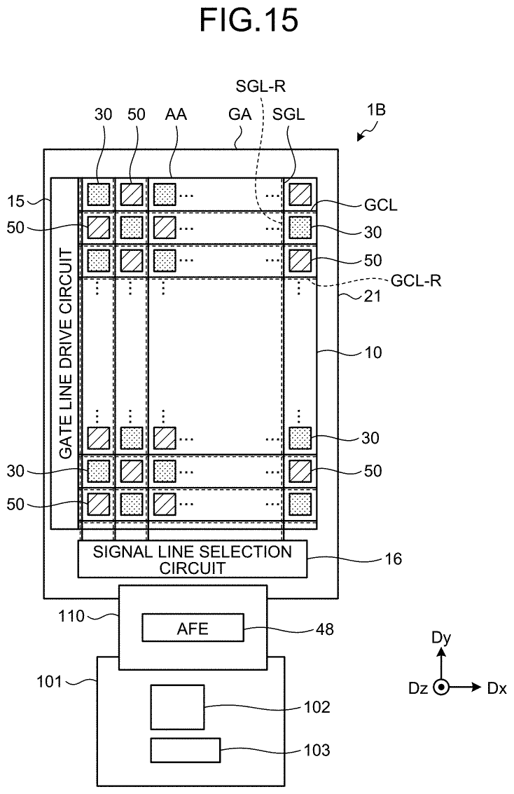

FIG. 15 is a plan view illustrating a detection device according to a third embodiment. As illustrated in FIG. 15, a detection device 1B of the third embodiment includes a plurality of the second optical sensors 50. The first optical sensors 30 and the second optical sensors 50 are provided in the detection area AA. In the detection area AA, the first optical sensors 30 and the second optical sensors 50 are alternately arranged along the first direction Dx and are also alternately arranged along the second direction Dy.

In other words, in the plan view from a direction orthogonal to the insulating substrate 21, the second optical sensor 50 is provided between the first optical sensors 30 adjacent in the first direction Dx. The second optical sensor 50 is provided between the first optical sensors 30 adjacent in the second direction Dy.

The gate lines GCL-R and the signal lines SGL-R are provided in the detection area AA along the gate lines GCL and the signal lines SGL, respectively. The gate lines GCL-R are coupled to the gate line drive circuit 15. The signal lines SGL-R are coupled to the signal line selection circuit 16. The signal line selection circuit 16 may couple a selected signal line SGL-R of the signal lines SGL-R to the detection circuit 48 in the same manner as in the case of the signal line SGL.

In the present embodiment, the second optical sensor 50 for reference corresponds to each of the first optical sensors 30. Therefore, the change in the first optical sensors 30 with time can be accurately monitored. Since the gate line drive circuit 15 and the signal line selection circuit 16 can be commonly used also as the drive circuit of the second optical sensors 50, the circuit scale of the surrounding circuit can be reduced. Since the second optical sensors 50 are arranged in a matrix having a row-column configuration in the detection area AA, the second detection signals Vdet-R may be used for detecting the biological information.

In FIG. 15, the first optical sensors 30 and the second optical sensors 50 are alternately arranged one by one in the first direction Dx. However, the arrangement is not limited thereto. One second optical sensor 50 may be provided for a plurality (for example, two to several tens) of the first optical sensors 30.

### Fourth Embodiment

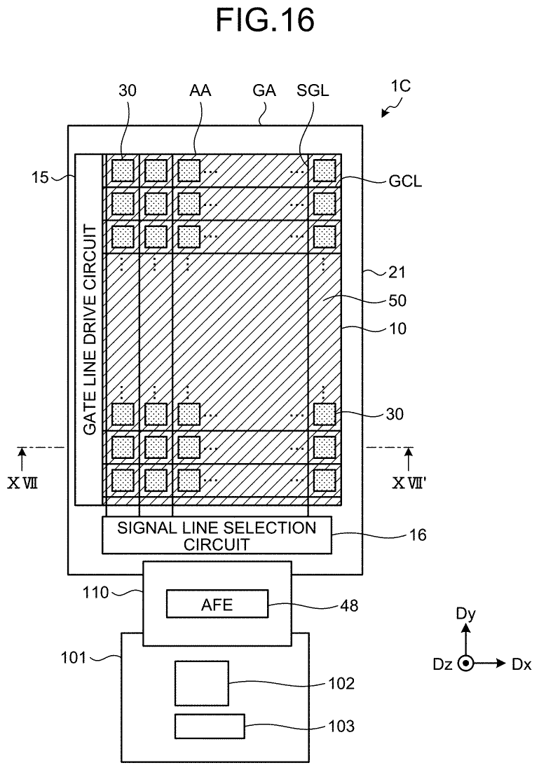

FIG. 16 is a plan view illustrating a detection device according to a fourth embodiment. As illustrated in FIG. 16, a detection device 1C of the fourth embodiment includes one second optical sensor 50 provided in the detection area AA. More specifically, the second optical sensor 50 is provided so as to cover the entire area of the detection area AA. The first optical sensors 30 are arranged in a matrix having a row-column configuration so as to overlap the one second optical sensor 50. The gate lines GCL and the signal lines SGL provided corresponding to the first optical sensors 30 are also arranged so as to overlap the one second optical sensor 50.

The second optical sensor 50 may be coupled to at least one of the gate line drive circuit 15 and the signal line selection circuit 16. Alternatively, the second optical sensor 50 may be electrically coupled to the detection circuit 48 and the control circuit 102 through coupling wiring provided in the peripheral area GA and not through the gate line drive circuit 15 and the signal line selection circuit 16.

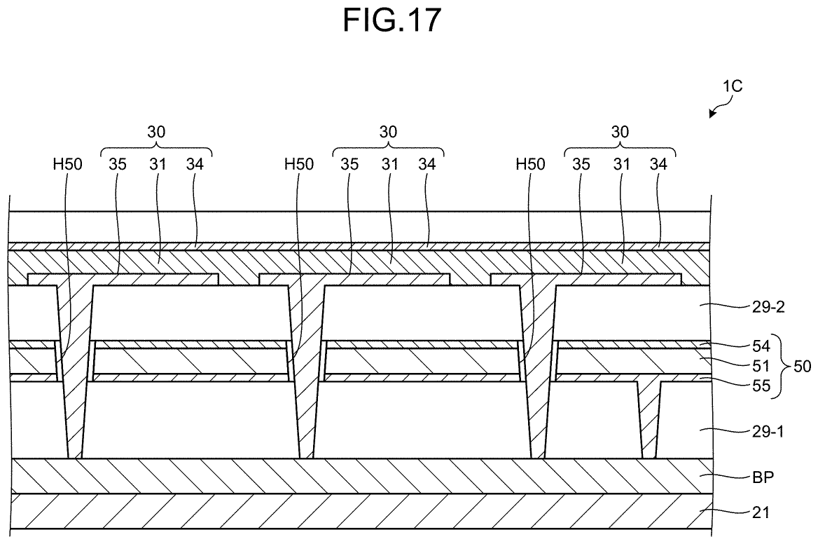

FIG. 17 is a XVII-XVII′ sectional view of FIG. 16. FIG. 17 is a sectional view illustrating a portion of the detection device 1C in an enlarged manner. While FIG. 17 illustrates the configuration of the backplane BP in a simplified manner, the backplane BP is provided with the first switching elements Tr corresponding to the first optical sensors 30 in the same manner as in FIG. 7. The backplane BP is also provided with the fifth switching element TrA corresponding to the second optical sensor 50.

As illustrated in FIG. 17, the first optical sensors 30 and the second optical sensor 50 are provided on the same insulating substrate 21. The first optical sensors 30 are provided on the upper side of the second optical sensor 50. More specifically, the second optical sensor 50 is provided on the upper side of a first smooth layer 29-1. The cathode electrode 55, the semiconductor layer 51, and the anode electrode 54 are stacked in this order on the first smooth layer 29-1. The cathode electrode 55 is coupled to the backplane BP through a contact hole passing through the first smooth layer 29-1.

A second smooth layer 29-2 is provided so as to cover the second optical sensor 50. The first optical sensors 30 are provided on the upper side of the second smooth layer 29-2. The cathode electrodes 35, the active layer 31, and the anode electrode 34 are stacked in this order on the second smooth layer 29-2. The cathode electrodes 35 are arranged so as to be separated on a first optical sensor 30 basis. That is, the cathode electrodes 35 are arranged in a matrix having a row-column configuration in the plan view. The active layer 31 and the anode electrode 34 are continuously provided so as to cover the cathode electrodes 35.

The second optical sensor 50 is provided with openings H50 in positions overlapping the respective first optical sensors 30. The cathode electrodes 35 of the first optical sensors 30 are coupled to the backplane BP through the second smooth layer 29-2, the openings H50, and the contact hole passing through the first smooth layer 29-1.

The above-described configuration allows the second optical sensor 50 to detect light that has passed through the first optical sensors 30. Since the second optical sensor 50 is provided in the entire detection area AA, the sensitivity of the second optical sensor 50 as a whole can be improved even when the amount of the light passing through each of the first optical sensors 30 is small. Since the first optical sensors 30 are provided so as to overlap the second optical sensor 50, the arrangement of the first optical sensors 30 in the plan view is less limited. That is, even when the second optical sensor 50 is provided in the detection area AA, the detection device 1C can ensure the light-receiving area of the first optical sensors 30 or can ensure the resolution of the first optical sensors 30.

### (Modification)

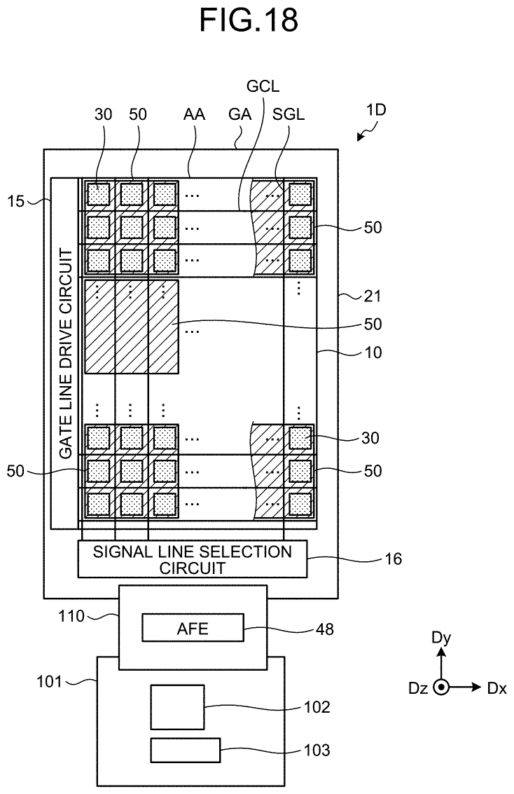

FIG. 18 is a plan view illustrating a detection device according to a modification of the fourth embodiment. A detection device 1D according to the modification of the fourth embodiment includes a plurality of the second optical sensors 50 provided in the detection area AA. The second optical sensors 50 are arranged in a matrix having a row-column configuration in the detection area AA. A plurality of the first optical sensors 30 are arranged in a matrix having a row-column configuration so as to overlap a corresponding one of the second optical sensors 50. In the example illustrated in FIG. 18, nine first optical sensors 30 are provided so as to overlap the corresponding one of the second optical sensors 50. However, the present disclosure is not limited to this configuration. Ten or more of the first optical sensors 30, such as several tens of the first optical sensors 30, may be provided so as to overlap a corresponding one of the second optical sensors 50.

Although the preferred embodiments of the present disclosure have been described above, the present disclosure is not limited to the embodiments described above. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure.

What is claimed is:

1. A detection device comprising:
a substrate;
a plurality of first optical sensors provided in a detection area of the substrate and comprising an organic material layer having a photovoltaic effect; and
at least one or more second optical sensors provided on the substrate and comprising an inorganic material layer having a photovoltaic effect.

2. The detection device according to claim 1, wherein
the first optical sensors are arranged in a matrix having a row-column configuration in the detection area, and
the second optical sensor or one of the second optical sensors is disposed in a peripheral area of the substrate.

3. The detection device according to claim 1, comprising a plurality of the second optical sensors, wherein
the first optical sensors are arranged in a matrix having a row-column configuration in the detection area, and
the second optical sensors are provided in a peripheral area of the substrate and are arranged along at least one side of the detection area.

4. The detection device according to claim 1, comprising a plurality of the second optical sensors, wherein
the first optical sensors and the second optical sensors are alternately arranged along a first direction in the detection area.

5. The detection device according to claim 1, wherein
the second optical sensor is or the second optical sensors are provided in the detection area, and
the first optical sensors are provided so as to overlap the second optical sensor or one of the second optical sensors.

6. The detection device according to claim 1, wherein the inorganic material layer is an inorganic semiconductor layer formed of amorphous silicon.

7. The detection device according to claim 1, comprising a control circuit configured to control detection in the first optical sensors and the second optical sensor or the second optical sensors, wherein
the control circuit is configured to control the detection in the first optical sensors based on a change in a difference signal between a first detection signal output from each of the first optical sensors and a second detection signal output from the second optical sensor or each of the second optical sensors.
